(19) United States
(12) Patent Application Publication
(10) Pub. No.: US 2010/0204309 A1
(43) Pub. Date: Aug. 12, 2010

(54) OLIGONUCLEOTIDE COMPOSITIONS FOR THE TREATMENT OF ALZHEIMER'S DISEASE

(75) Inventors: Sébastien Hérbert, Québec (CA); Bart De Strooper, Leuven (BE)

Correspondence Address: TRASKBRITT, P.C., P.O. BOX 2550, SALT LAKE CITY, UT, 84110, US

(73) Assignees: VIB VZW, Gent (BE); KATHOLIEKE UNIVERSITEIT LEUVEN, K.U. LEUVEN R & D, Leuven (BE)

(21) Appl. No.: 12/450,698

(22) PCT Filed: Apr. 21, 2008
(86) PCT No.: PCT/EP2008/054787

§ 371 Date: Feb. 26, 2010

(30) Foreign Application Priority Data
Apr. 19, 2007 (EP) 07106482.8

(51) Int. Cl. A61K 31/7088; C12Q 1/68; C07H 21/04; A61P 25/28

(52) U.S. Cl. 514/44 R; 435/6; 536/23.1

(57) ABSTRACT

The invention relates to the identification of microRNAs whose expression is diminished in neurodegenerative disorders. In particular, the identified microRNAs can be used for the downregulation of the beta-secretase (BACE1) in mammalian brain and hence for the treatment of Alzheimer's disease.

### TECHNICAL FIELD

The invention relates generally to biotechnology, and, more particularly, to the identification of microRNAs the expression of which is diminished in neurodegenerative disorders. In particular, the identified microRNAs can be used for the downregulation of beta-secretase (BACE1) in mammalian brain, and hence has applicability for the treatment of Alzheimer's disease (“AD”).

### BACKGROUND

MicroRNAs (miRNAs) are an abundant class of newly identified endogenous non-protein-coding small RNAs with 20-25 nucleotides in length. A majority of identified miRNAs are highly evolutionarily conserved among many distantly related species, some from worms to human in animals, and mosses to higher plants, suggesting that miRNAs play a very important role in essential biological processes, including developmental timing, stem cell differentiation, signaling transduction, disease, and cancer. The non-coding miRNAs suppress gene expression via imperfect complementary binding to the 3′UTR of target mRNAs leading to their translational repression and sometimes degradation (Ambros, 2004; Bartel, 2004). Several miRNAs are specifically expressed in the brain (Barad et al., 2004; Miska et al., 2004; Sempere et al., 2004) and some have been associated with neuronal differentiation, synaptic plasticity and memory formation (Mehler and Mattick, 2006).

### DISCLOSURE OF THE INVENTION

We have identified specific miRNAs that are downregulated in sporadic AD brains which are involved in the regulation of the beta-secretase APP cleaving enzyme 1 (BACE1). BACE1 is a known target in Alzheimer's disease (AD) (Fukumoto et al., 2002; Holsinger et al., 2002; Sun et al., 2002; Yang et al., 2003). The miRNAs can be used for the manufacture of a medicament to treat Alzheimer's disease.

### BRIEF DESCRIPTION OF THE FIGURES

FIG. 1: BACE1 upregulation in sporadic AD brain.

(A) Representative Western blot analysis of BACE1, Nicastrin and Pen-2 in AD and age-matched controls is shown. β-Actin was used as normalization control. (B) Densitometric quantifications of BACE1 protein levels in sporadic AD (n=34) versus controls (n=21). For each gel, the average of the controls was used as reference (i.e., 1 fold). (C) Quantitative RT-PCR of BACE1 mRNA from representative controls (n=5) and from AD patients (n=5). Densitometric quantifications of BACE1 protein levels (as in [B]) from the same samples are shown here for comparison. β-Actin mRNA and protein were used as normalization controls. NS, non-specific band.

FIG. 2. BACE1 is a novel miRNA target gene.

(A) List of candidate miRNAs used in this study identified by various algorithms: miRanda (microrna.org), Targetscan (targetscan.org) or Pictar (pictar.bio.nyu.edu). “X” indicates a positive hit in the algorithm. (B) Schematic representation (not to scale) of the BACE1 3′UTR luciferase construct used in this study. The “start” and “stop” codons of the Luc ORF are indicated. The sequences as well as the putative binding sites of the miR-29a/b-1 family are shown (SEQ ID NO:1 and 2, respectively). In the hBACE1 3′UTR Mut construct (see, SEQ ID NO: 12 and 13), the binding site for miR-29 is mutated as indicated. (C) BACE1 3′UTR (wt or mut) luciferase and Renilla luciferase constructs were transfected into HeLa cells with the indicated oligonucleotides at a final concentration of 75 nM. Normalized sensor luciferase activity is shown as a percentage of the control with a scrambled oligonucleotide. Error bars represent standard deviations derived from three or more independent experiments. Statistical significance between control (scrambled miR-treated) and candidate miR-treated Hela cells was determined by a Wilcoxon test.

FIG. 3. Misregulation of miR-29a/b-1 in AD brain.

(A)(B) Quantitative RT-PCR of miR29a and miR29b-1 in controls (n=21), AD patients with low BACE1 (n=23), AD patients with high BACE1 (n=11) and non-AD dementia patients (n=9). Relative expression is shown as percentage using the mean of the control group as reference (i.e., 100%). (C) Quantitative RT-PCR of miR-29c in controls (n=21), AD patients with low BACE1 (n=21), AD patients with high BACE1 (n=9). Relative expression is shown as percentage using the controls group as reference (i.e., 100%). (D) Significant correlation between BACE1 protein and miR-29a in AD patients (n=34) as assessed by quantitative RT-PCR. Ubiquitously expressed miR-16 was used as normalization control for all experiments. Every data point reflects the mean of three independent RT reactions.

FIG. 4. Modulation of BACE1 activity and Aβ levels by miR-29a/b-1.

(A) Western blot analysis of endogenous APP-CTFs (α and β) in pre-miR (miR-29a/b-1) or anti-miR (miR-29a/b-1 antisense)-treated HEK293-APP Swedish cells. Quadruplicate samples are shown. APP-CTFα as well as β-Actin were used as loading controls.

FIG. 5.

Conservation plot of human BACE1 3′UTR with putative miRNA binding sites. The percentage of conservation in 5 mammalian species (human, chimp, mouse, rat, and dog) is shown.

FIG. 6.

Relative expression levels of various miRNAs in the adult brain of controls (n=3, ages 70 years old −/+19). The ubiquitously expressed miR-16 was used as normalization control. The weakly expressed miR-210 was used as calibrator (i.e., used as reference to determine 1 fold baseline). MiR-19b (which is co-expressed as a cluster with miR-19a, miR-17-5p and miR-20a[http://microma.sanger.ac.uk]) is expressed at moderate levels. MiR-124a was used as a brain-enriched miRNA control.

FIG. 7.

Quantitative RT-PCR of miR-9 in controls (n=20), AD patients with low BACE1 (n=16), AD patients with high BACE1 (n=9) and non-AD dementia patients (n=9). Relative expression is shown as percentage using the controls group as reference (i.e., 100%). Statistical significance was determined by a Mann-Whitney test.

TABLE 1
Neuropathological and biochemical analysis of human brain samples used in our study.

| | Sample | Age | Sex | PMD(h) | Apoe | Tau staging | Amyloid staging | [symbol] ynuclein stagin | Clinical diagnostic | Pathology |
|---|---|---|---|---|---|---|---|---|---|---|
| Control | Ml.82 | 82 | 2 | — | E3/E3 | S3 | S1, S0 | Kos.0 | Ctrl | control |
| n = 21 | Dp.69 | 69 | 1 | — | E2/E3 | S4 | S0, | — | Ctrl | control |
| | Ci.51 | 51 | 2 | — | E2/E3 | S3 | S0, S0 | — | Ctrl | control |
| | Dv.77 | 77 | 1 | — | E2/E3 | S3 | S5, S0 | — | Ctrl | control |
| | Ls.65 | 65 | 2 | 24 | E3/E4 | S0 | S0, S0 | — | Ctrl | control |
| | Dc.26 | 26 | 2 | 15 | E3/E4 | S0 | S0, S0 | — | Ctrl | control |
| | Bu.72 | 72 | 1 | 12 | E2/E3 | <S3 | S0, S0 | — | Ctrl (chimiotherapy) | control |
| | Rc.42 | 42 | 1 | — | E3/E4 | <S4 | S0, S0 | — | Ctrl | control |
| | Co.72 | 72 | 2 | 19 | E3/E3 | S2 | S0, S0 | — | Ctrl | control |
| | Cu.43 | 43 | 1 | — | E3/E3 | S0 | S1, S0 | — | Ctrl (ethylism) | control |
| | Bn.89 | 89 | 2 | 16 | E2/E3 | S2 | S0, S0 | — | Ctrl | control |
| | Dm.46 | 46 | 2 | 24 | E2/E3 | S0 | S0, S0 | — | vascular | control |
| | Ru.78 | 78 | 2 | 24 | E2/E3 | S1 | S0, S0 | — | Ctrl | control |
| | Zu.13 | 13 | 1 | 20 | — | S0 | S0, S0 | — | Ctrl | control |
| | Mi.45 | 45 | 1 | — | E3/E3 | <S4 | S0, S0 | — | coma, clonic crisis | control |
| | Go.59 | 59 | 1 | — | E3/E4 | S0 | not analyzed | Kos.0 | vascular dementia | control |
| | Hn.95 | 95 | 1 | 60 | E2/E3 | S4 | S0, S0 | — | Ctrl, cardiovascular | control |
| | | | | | | | | | failure | |
| | Bi.93 | 93 | 2 | — | E3/E3 | S4 | S1, S0 | — | Ctrl, pneumopathy | control |
| | Mi.88 | 88 | 1 | 13 | E3/E3 | S6 | S2, | — | Ctrl | control |
| | Gr.68 | 68 | 2 | 18 | E2/E3 | <S3 | S0, S0 | — | Ctrl, encephalitis | control |
| | Bu.44 | 44 | 1 | — | E3/E4 | S3 | S0, S0 | — | peritonite broncho. | control |
| | Ge.66 | 66 | 2 | 56 | E3/E3 | S3 | S0, S0 | Kos.0 | MCI | control |
| | Cr.65 | 65 | 2 | — | E3/E3 | S3 | S1, S0 | — | encephalitis | control |
| Alzheimer | Fi.74 | 74 | 1 | 4 | E4/E4 | S10 | S9, S8 | Kos.0 | Pr Alz | Def Alz |
| n = 34 | Dp.83 | 83 | 2 | 5 | E3/E3 | S10 | S7, | Kos.1 | Pr Alz | Def Alz |
| | Mz.69 | 69 | 1 | 10 | E2/E4 | S10 | S8, S7 | — | pr semantic dementia | Def Alz |
| | Vn.64 | 64 | 2 | — | E3/E4 | S7 | S7, S0 | Kos.2/Syn.1 | po Alz, po LBD, po | Def Alz |
| | | | | | | | | | Vasc | |
| | Hv.84 | 84 | 2 | 20 | E2/E3 | S9 | S6, S5 | Kos.0 | dementia | mixte dementia |
| | Qi.74 | 74 | 1 | 5 | E3/E4 | S9 | S7, S0 | Kos2 | pr CBD | Def Alz |
| | Wt.78 | 78 | 1 | 15 | E4/E4 | S10 | S7, S8 | — | po Alz + vascular | mixte dementia |
| | Cc.70 | 70 | 2 | — | E3/E3 | S9a | S4, S0 | — | pr Alz | Def Alz |
| | Vn.84 | 84 | 2 | 20 | E3/E4 | S10 | S7, S7 | — | pr Alz | Def Alz |
| | Vn.81 | 81 | 1 | 10 | E3/E3 | S10 | S5, S0 | — | pr Alz, Pr LBD | Def Alz |
| | Fs.69 | 69 | 1 | 6 | E3/E4 | S10 | S8, S0 | Kos.0 | po Alz, +vascular | Def Alz |
| | Df.73 | 73 | 1 | 9 | E3/E3 | S10 | S7, S0 | Kos.0 | po Alz; po LBD | Def Alz |
| | Pr.71 | 71 | 2 | 6 | E4/E4 | S10 | S9, S3 | Kos.9/Syn.8 | po Alz; po LBD | LBD, AD+ |
| | Mt.72 | 72 | 1 | 18 | E3/E4 | S10 | S9, S3 | Kos.1 | pr Alz | Def Alz |
| | Du.90 | 90 | 2 | 1 | E3/E4 | S10 | S8, S3 | — | def Alz | Def Alz |
| | Cu.55 | 55 | 2 | 12 | E3/E3 | S10 | S7, | — | pr Alz | Def Alz |
| | Cu.70 | 70 | 2 | 6 | E3/E3 | S10 | S8, S5 | — | pr Alz | Def Alz |
| | Ao.79 | 79 | 1 | 22 | E3/E4 | S10 | S8, S0 | — | pr Alz | Def Alz |
| | Fu.63 | 63 | 2 | 5 | E3/E4 | S10 | S8, S3 | — | pr Alz, vascular | Def Alz |
| | Pn.70 | 70 | 1 | 24 | E4/E4 | S10 | S9, S7 | — | pr Alz | Def Alz |
| | Dm.83 | 83 | 2 | — | E3/E4 | S10 | S8, S0 | — | pr Alz, vascular | Def Alz |
| | Gn.67 | 67 | 1 | — | E3/E4 | S10 | S8, S0 | — | pr Alz | Def Alz |
| | To.77 | 77 | 2 | 24 | E3/E4 | S10 | S8, S5 | — | pr Alz | Def Alz |
| | Dn.57 | 57 | 1 | 28 | E3/E4 | S10 | S9, S4 | — | pr Alz | Def Alz |
| | Di.75b | 75 | 2 | 4 | E3/E3 | S10 | S8, S4 | — | pr Alz | Def Alz |
| | Nv.78 | 78 | 2 | — | E3/E4 | S10 | S8, S4 | — | pr Alz, vascular | Def Alz |
| | Fa.85 | 85 | 2 | 5 | E3/E3 | S10 | S8, S8 | Kos.0 | pr Alz, vascular | Def Alz |
| | Mr.73 | 73 | 1 | — | E4/E4 | S10 | S8, S6 | Kos.0 | pr Alz | Def Alz |
| | Vn.78 | 78 | 2 | 5 | E2/E3 | S10 | S8, S8 | 0 | pr Alz, AA | Def Alz |
| | Gs.86 | 86 | 2 | 61 | E2/E3 | S9a | S8, S8 | — | pr Alz | Def Alz |
| | Ci.65 | 65 | 2 | 6 | E3/E3 | S9b | S9, S4 | — | po Alz | Def Alz |
| | Dq.80 | 80 | 1 | 12 | E3/E4 | S10 | S9, S0 | — | pr Alz, vascular | Def Alz |
| | Se.69 | 69 | 1 | 24 | E3/E4 | S10 | S10, S5 | — | pr Alz | Def Alz |
| Non-AD | Gs.71 | 71 | 1 | — | E3/E3 | Type II | not analyzed | — | PSP | non-AD |
| n = 9 | Bn.75 | 75 | 2 | — | E2/E3 | Type II | not analyzed | — | CBD | non-AD |
| | Ln.80 | 80 | 1 | — | E3/E3 | Type II | S0, S0 | 0 | CBD, sub-cortical | non-AD |
| | Ui.58 | 58 | 1 | — | E2/E3 | Type II | not analyzed | — | PSP | non-AD |
| | Pp.68 | 68 | 1 | — | E2/E3 | Type II | not analyzed | — | PSP | non-AD |
| | Oj. | | | — | E2/E3 | Type II | not analyzed | — | PSP | non-AD |
| | Cr.66 | 66 | 2 | — | E3/E3 | Type II | S0, S0 | — | CBD | non-AD |
| | Br.83 | 83 | 1 | 21 | — | Type II | S4, S0 | — | | non-AD |
| | Gu.81 | 81 | 2 | 13 | E3/E3 | Type V | S4, S0 | — | Tauopathy type V | non-AD |

All samples are taken from the frontal pole cortex.
Def Alz = definite Alzheimer according to the NIA/Reagan criteria, 1997
LBD, AD+ = Definite Alzheimer associated with LBD (neocortical Lewy [symbol]
mixte dementia: Definite Alzheimer + vascular pathology
Kos = Kosaka criteria (number of Lewy bodies in different areas)
synuclein staging: according to Deramecourt et al, 2006, JNEN
Po, Pr, Def: possible, probable, definite
PSP: Progressive Supranuclear Palsy
LBD: Lewy body disease
CBD: corticobasal degeneration
NIA: National Institute of Aging [symbol]
indicates data missing or illegible when filed

### DETAILED DESCRIPTION OF THE INVENTION

It has been shown in the art that AD patients display an increased BACE1 protein expression in the brain (Fukumoto et al., 2002; Holsinger et al., 2002; Sun et al., 2002; Yang et al., 2003) pointing to the fact that BACE1 is an important drug target for the treatment of AD. In the present invention we also concluded that about 30% of sporadic AD patients have an elevated BACE1 expression. Based on 1) in vitro validation in cell cultures and 2) careful analysis of a series of well preserved frozen brain tissue of a sporadic AD population, we identify miR-29a/b-1 as a significant suppressor of BACE1 protein expression. In a representative set of sample of patients investigated, BACE1 mRNA was not significantly changed, while regression analysis indicated that changes in miR-29a contributed significantly (p<0.03) to overall BACE1 expression in this sporadic AD population (34 patients). We speculate that during aging the gene regulatory network provided by miRNAs could become progressively compromised in some individuals. This could influence brain function in many ways, but if by chance the miR-29a/b-1 cluster is affected, this leads to decreased suppression of BACE1 expression and increased Aβ generation.

In a first embodiment, the invention relates to an oligonucleotide of less than 100 nucleotides comprising SEQ ID NO: 1 and/or SEQ ID NO: 2 or a modified oligonucleotide of less than 100 nucleotides comprising a modified SEQ ID NO: 1 and/or SEQ ID NO: 2 for use as a medicament.

In another embodiment, the invention relates to the use of an oligonucleotide of less than 100 nucleotides comprising SEQ ID NO: 1 and/or SEQ ID NO: 2 or a modified oligonucleotide of less than 100 nucleotides comprising a modified SEQ ID NO: 1 and/or SEQ ID NO: 2 for the manufacture of a medicament to treat Alzheimer's disease.

In another embodiment, the invention relates to an oligonucleotide of less than 100 nucleotides comprising SEQ ID NO: 1 and/or SEQ ID NO: 2 or a modified oligonucleotide of less than 100 nucleotides comprising a modified SEQ ID NO: 1 and/or SEQ ID NO: 2 for the treatment of Alzheimer's disease.

In a particular embodiment, the oligonucleotide or modified oligonucleotide is less than 90, less than 80, less than 70, less than 60, less than 50 or even less than 40 nucleotides.

The microRNA sequences are depicted in SEQ ID NO: 1 and 2 of the accompanying SEQUENCE LISTING. SEQ ID NO: 1 depicts the mature sequence of the human miR-29a and SEQ ID NO: 2 depicts the mature sequence of human miR29-b-1. The murine microRNA counterparts have an identical sequence as SEQ ID NO: 1 and 2. SEQ ID NO: 1 and 2 are also designated in the present invention as anti-BACE microRNAs because SEQ ID NO: 1 and 2 are able to reduce the activity of BACE1 in a cell or organ.

In another embodiment, the invention relates to the use of modified anti-BACE microRNA molecules.

In yet another embodiment, the invention envisages the use of chemically modified anti-BACE microRNA molecules.

In yet another embodiment, the anti-BACE microRNA molecules are single stranded.

In yet another embodiment, the anti-BACE microRNA molecules are double stranded.

In yet another embodiment, the invention relates to a method for inhibiting BACE1 activity in a cell, organ, or organism. In the present invention, BACE refers to BACE1, both terms are herein used interchangeably. The method comprises introducing into a cell, an organ or an organism an oligonucleotide comprising an anti-BACE microRNA molecule.

In yet a further embodiment, the invention relates to a method for treating Alzheimer's disease in a mammal in need thereof. The method comprises introducing into the mammal an effective amount of an anti-BACE microRNA molecule. In a preferred embodiment, a mammal is human.

MicroRNA molecules are known in the art (see, for example, Bartel, Cell, 2004, 116, 281-297 for a review on microRNA molecules). The definitions and characterizations of microRNA molecules in the article by Bartel are hereby incorporated by reference. Such molecules are derived from genomic loci and are produced from specific microRNA genes. Mature microRNA molecules are processed from precursor transcripts that form local hairpin structures. The hairpin structures are typically cleaved by an enzyme known as Dicer, generating thereby one microRNA duplex. See the above reference by Bartel.

In yet another embodiment, the invention relates to a DNA or RNA molecule comprising a sequence shown in SEQ ID NO:1 or 2, and equivalents thereof. Preferably, the DNA or RNA molecule comprises at least ten, at least thirteen, more preferably at least fifteen, and even more preferably at least twenty contiguous bases having a sequence of bases in an anti-BACE microRNA shown in SEQ ID NO: 1 or 2.

As used herein, a base refers to any one of the nucleotide bases normally found in naturally occurring DNA or RNA. The bases can be purines or pyrimidines. Examples of purine bases include adenine (A) and guanine (G). Examples of pyrimidine bases include thymine (T), cytosine (C) and uracil (U). The adenine can be replaced with 2, 6-diaminopurine. Sequences of unmodified nucleic acid molecules disclosed in this specification are shown having uracil bases. Uracil bases occur in unmodified RNA molecules. The invention also includes unmodified DNA molecules. The sequence of bases of the unmodified DNA molecule is the same as the unmodified RNA molecule, except that in the unmodified DNA molecule, the uracil bases are replaced with thymine bases. Each base in the sequence can form a Watson-Crick base pair with a complementary base. Watson-Crick base pairs as used herein refer to the hydrogen bonding interaction between, for example, the following bases: adenine and thymine (A-T); adenine and uracil (A-U); and cytosine and guanine (C-G).

Equivalents refer to molecules wherein up to thirty percent of the at least ten contiguous bases are wobble bases, and up to ten percent, and preferably up to five percent of the contiguous bases are non-complementary. As used herein, wobble base refer to either: 1) substitution of a cytosine with a uracil, or 2) the substitution of an adenine with a guanine, in the sequence of the molecule. These wobble base substitutions are generally referred to as UG or GU wobbles. The term “non-complementary” as used herein refers to additions, deletions, mismatches or combinations thereof. Additions refer to the insertion in the contiguous sequence of any base described above. Deletions refer to the removal of any moiety present in the contiguous sequence. Mismatches refer to the substitution of one of the bases in the contiguous sequence with a different base. The additions, deletions or mismatches can occur anywhere in the contiguous sequence, for example, at either end of the contiguous sequence or within the contiguous sequence of the molecule. Typically, the additions, deletions or mismatches occur at the end of the contiguous if the contiguous sequence is relatively short, such as, for example, from about ten to about fifteen bases in length. If the contiguous sequence is relatively long, such as, for example, a minimum of sixteen contiguous sequences, the additions, deletions, or mismatches may occur anywhere in the contiguous sequence. For example, none or one of the contiguous bases may be additions, deletions, or mismatches when the number of contiguous bases is ten to nineteen; and a maximum of one or two additions, deletions, or mismatches are permissible when the number of contiguous bases is twenty to twenty-three. In addition to the at least ten contiguous nucleotides of the anti-BACE microRNA, the isolated DNA or RNA molecule may also have one or more additional nucleotides. There is no upper limit to the additional number of nucleotides. Typically, no more than about 500 nucleotides, and preferably no more than about 300 nucleotides are added to the at least ten contiguous bases of an anti-BACE microRNA. Any nucleotide can be added. The additional nucleotides can comprise any base described above. Thus, for example, the additional nucleotides may be any one or more of A, G, C, T, or U.

In one embodiment, the anti-BACE microRNA is part of a hairpin precursor sequence or fragment thereof. The anti-BACE microRNA or hairpin precursor can be inserted into a vector, such as, for example, a recombinant vector. Typically, to construct a recombinant vector containing an anti-BACE microRNA, the hairpin precursor sequence which contains the anti-BACE microRNA sequence is incorporated into the vector.

The recombinant vector may be any recombinant vector, such as a plasmid, a cosmid or a phage. Recombinant vectors generally have an origin of replication. The vector may be, for example, a viral vector, such as an adenovirus vector or an adeno-associated virus (AAV) vector. The vector may further include a selectable marker. Suitable selectable markers include a drug resistance marker, such as tetracycline or gentamycin, or a detectable gene marker, such as beta-galactosidase or luciferase.

Preferably, the molecule is essentially pure, which means that the molecules are free not only of other nucleic acids, but also of other materials used in the synthesis and isolation of the molecule. Materials used in synthesis include, for example, enzymes. Materials used in isolation include, for example, gels, such as SDS-PAGE. The molecule is at least about 90% free, preferably at least about 95% free and, more preferably at least about 98% free of such materials.

The sequence of bases in a microRNA or hairpin precursor is highly conserved. Due to the high conservation, the sequence can be derived from a cell (e.g., a neuron) of any mammal. Examples of mammals include pet animals, such as dogs and cats, farm animals, such as cows, horses and sheep, laboratory animals, such as rats, mice and rabbits, and primates, such as monkeys and humans. Preferably, the mammal is human or mouse.

Modified Anti-BACE microRNA Molecules

In another embodiment, the invention relates to a modified anti-BACE microRNA molecule. The modified microRNA molecule can be any of the anti-BACE microRNA molecules, hairpin precursor molecules, or equivalents thereof described above, except that the modified molecule comprises at least one modified moiety (i.e., at least one moiety is not an unmodified deoxyribonucleotide moiety or ribonucleotide moiety). In this embodiment, the modified anti-BACE microRNA molecule comprises a minimum number of ten moieties, preferably a minimum of thirteen, more preferably a minimum of fifteen, even more preferably a minimum of eighteen, and most preferably a minimum of twenty-one moieties.

Each modified moiety comprises a base bound to a backbone unit. The backbone unit may be any molecular unit that is able to stably bind to a base and to form an oligomeric chain. In this specification, the backbone units of a modified moiety do not include the backbone units commonly found in naturally occurring DNA or RNA molecules. Typically, such modified anti-BACE microRNA molecules have increased nuclease resistance. Therefore, the nuclease resistance of the molecule is increased compared to a sequence containing only unmodified ribonucleotide moieties, unmodified deoxyribonucleotide moieties or both. Such modified moieties are well known in the art, and were reviewed, for example, by Kurreck, Eur. J. Biochem. 270, 1628-1644 (2003).

A modified moiety can occur at any position in the anti-BACE microRNA molecule. For example, anti-BACE microRNA molecules can have at least one modified moiety at the 3′ end of the molecule and preferably at least two modified moieties at the 3′ end, the anti-BACE microRNA molecules can also have at least one modified moiety and preferably at least two modified moieties at the 5′ end of the molecule. The anti-BACE microRNA molecules can also have at least one and preferably at least two modified moieties between the 5′ and 3′ end of the molecule to increase resistance of the molecule to endonucleases. Preferably, at least about 10%, more preferably at least about 25%, even more preferably at least about 50%, and further more preferably at least about 75%, and most preferably at least about 95% of the moieties are modified. In one embodiment, all of the moieties are modified (e.g., nuclease resistant).

In one example of a modified anti-BACE microRNA molecule, the molecule comprises at least one modified deoxyribonucleotide moiety. Suitable modified deoxyribonucleotide moieties are known in the art. Such modified deoxyribonucleotide moieties comprise, for example, phosphorothioate deoxyribose groups as the backbone unit.

Another suitable example of a modified deoxyribonucleotide moiety is an N′3-N′5 phosphoroamidate deoxyribonucleotide moiety, which comprises an N′3-N′5 phosphoroamidate deoxyribose group as the backbone unit.

In another example of a modified anti-BACE microRNA molecule, the molecule comprises at least one modified ribonucleotide moiety. A suitable example of a modified ribonucleotide moiety is a ribonucleotide moiety that is substituted at the 2′ position.

Another suitable example of a substituent at the 2′ position of a modified ribonucleotide moiety is a C1 to C4 alkoxy-C1 to C4 alkyl group. The C1 to C4 alkoxy (alkyloxy) and C1 to C4 alkyl group may comprise any of the alkyl groups described above. The preferred C1 to C4 alkoxy-C1 to C4 alkyl group is methoxyethyl.

Another suitable example of a modified ribonucleotide moiety is a ribonucleotide that has a methylene bridge between the 2′-oxygen atom and the 4′-carbon atom.

Another suitable example of a modified ribonucleotide moiety is a ribonucleotide that is substituted at the 2′ position with fluoro group. Such 2′-fluororibonucleotide moieties are known in the art.

In another example of a modified anti-BACE microRNA molecule, the molecule comprises at least one modified moiety comprising a base bound to an amino acid residue as the backbone unit. Modified moieties that have at least one base bonded to an amino acid residue will be referred to herein as peptide nucleic acid (PNA) moieties. Such moieties are nuclease resistance, and are known in the art. The amino acids can be any amino acid, including natural or non-natural amino acids. Naturally occurring amino acids include, for example, the twenty most common amino acids normally found in proteins, i.e., alanine (Ala), arginine (Arg), asparagine (Asn), aspartic acid (Asp), cysteine (Cys), glutamine (Glu), glutamic acid (Glu), glycine (Gly), histidine (His), isoleucine (Ileu), leucine (Leu), lysine (Lys), methionine (Met), phenylalanine (Phe), proline (Pro), serine (Ser), threonine (Thr), tryptophan, (Trp), tyrosine (Tyr), and valine (Val).

The non-natural amino acids may, for example, comprise alkyl, aryl, or alkylaryl groups. Some examples of alkyl amino acids include alpha-aminobutyric acid, beta-aminobutyric acid, gamma-aminobutyric acid, delta-aminovaleric acid, and epsilon-aminocaproic acid. Some examples of aryl amino acids include ortho-, meta-, and para-aminobenzoic acid. Some examples of alkylaryl amino acids include ortho-, meta-, and para-aminophenylacetic acid, and gamma-phenyl-beta-aminobutyric acid.

Non-naturally occurring amino acids also include derivatives of naturally occurring amino acids. The derivative of a naturally occurring amino acid may, for example, include the addition or one or more chemical groups to the naturally occurring amino acid.

In another example of a modified anti-BACE microRNA molecule, the molecule comprises at least one morpholino phosphoroamidate nucleotide moiety.

In a further example of a modified anti-BACE microRNA molecule, the molecule comprises at least one cyclohexene nucleotide moiety.

In a final example of a modified anti-BACE microRNA molecule, the molecule comprises at least one tricyclo nucleotide moiety.

The modified anti-BACE microRNA molecules of the invention comprise at least ten, preferably at least thirteen, more preferably at least fifteen, and even more preferably at least twenty contiguous bases having any of the contiguous base sequences of a naturally occurring anti-BACE microRNA molecule shown in SEQ ID NO:1-2. In a preferred embodiment, the modified anti-BACE microRNA molecules comprise the entire sequence of any of the anti-BACE microRNA molecule shown in SEQ ID NO: 1-2.

The modified anti-BACE microRNA molecules include equivalents thereof. Equivalents include wobble bases and non-complementary bases as described above. In yet another embodiment, caps can be attached to one end, both ends, and/or between the ends of the molecule in order to increase nuclease resistance of the modified anti-BACE microRNA molecules or unmodified isolated DNA or RNA molecules of the present invention described above to exonucleases. Any cap known to those in the art for increasing nuclease resistance can be employed. Examples of such caps include inverted nucleotide caps and chemical caps.

An inverted nucleotide cap refers to a sequence of nucleic acids attached to the anti-BACE microRNA molecule at the 5′ and/or the 3′ end. There is no limit to the maximum number of nucleotides in the inverted cap just as long as it does not interfere with binding of the anti-BACE microRNA molecule or isolated DNA or RNA molecule to its target mRNA. Any nucleotide can be used in the inverted nucleotide cap. Alternatively, a chemical cap can be attached to the 5′ end, to the 3′ end, to both ends of the molecule, and/or to any moiety(ies) between the 5′ end and 3′ end of the modified anti-BACE microRNA molecule or isolated DNA or RNA molecule in order to increase nuclease resistance to exonucleases and/or endonucleases. The chemical cap can be any chemical group known to those in the art for increasing nuclease resistance of nucleic acids. Examples of such chemical caps include hydroxyalkyl or aminoalkyl groups. Hydroxyalkyl groups are sometimes referred to as alkyl glycoyl groups (e.g., ethylene glycol). Aminoalkyl groups are sometimes referred to as amino linkers.

The alkyl chain in the hydroxyalkyl group or aminoalkyl groups can be a straight chain or branched chain. The minimum number of carbon atoms present in the alkyl chain is one, preferably at least two, and more preferably at least about three carbon atoms. The maximum number of carbon atoms present in the alkyl chain is about eighteen, preferably about sixteen, and more preferably about twelve. Typical alkyl groups include methyl, ethyl, and propyl. The alkyl groups can be further substituted with one or more hydroxyl and/or amino groups. Thus, in one aspect of the invention, the invention relates to a method for inhibiting BACE activity in a cell.

The anti-BACE microRNA molecules of the present invention are capable of inhibiting BACE1 activity by binding to the BACE1 messenger RNA (mRNA) in a host cell (or organ).

The microRNA molecules can be introduced into a cell by any method known to those skilled in the art. The method for inhibiting BACE activity in a cell comprises introducing into the cell an anti-BACE microRNA molecule.

For example, the microRNA molecules can be injected directly into a cell, such as by microinjection. Alternatively, the molecules can be contacted with a cell, preferably aided by a delivery system.

Useful delivery systems include, for example, liposomes and charged lipids. Liposomes typically encapsulate oligonucleotide molecules within their aqueous center. Charged lipids generally form lipid-oligonucleotide molecule complexes as a result of opposing charges.

These liposomes-oligonucleotide molecule complexes or lipid-oligonucleotide molecule complexes are usually internalized in cells by endocytosis. The liposomes or charged lipids generally comprise helper lipids which disrupt the endosomal membrane and release the oligonucleotide molecules.

Other methods for introducing a microRNA molecule into a cell include use of delivery vehicles, such as dendrimers, biodegradable polymers, polymers of amino acids, polymers of sugars, and oligonucleotide-binding nanoparticles. In addition, pluoronic gel as a depot reservoir can be used to deliver the microRNA oligonucleotide molecules over a prolonged period. The above methods are described in, for example, Hughes et al., Drug Discovery Today 6, 303-315 (2001); Liang et al. Eur. J. Biochem. 269 5753-5758 (2002); and Becker et al., In Antisense Technology in the Central Nervous System (Leslie, R. A., Hunter, A. J. & Robertson, H. A., eds.), pp. 147-157, Oxford University Press.

Targeting of a microRNA molecule or an anti-microRNA molecule to a particular cell can be performed by any method known to those skilled in the art. In a particular embodiment the microRNAs of the invention are targeted to hippocampal neurons. To this the microRNA molecule is for example conjugated to an antibody that is able to bind telencephalin. Alternatively the microRNA molecule is conjugated to a laminin derived peptide (Van Meerbergen B. et al (2007) J. of Exp. Nanoscience Vol. 2, pp. 101-114). The technology to direct molecules to hippocampal neurons is described in WO2006030013 (“The modulation of phagocytosis in neurons”).

In another embodiment, the invention provides a method for treating Alzheimer's disease in a mammal in need thereof. The method comprises introducing into the mammal an effective amount of an anti-BACE microRNA molecule having at least ten contiguous bases having a sequence shown in SEQ ID NO: 1 or 2. The effective amount is determined during pre-clinical trials and clinical trials by methods familiar to physicians and clinicians.

The anti-BACE microRNA molecules can be introduced into the mammal by any method known to those in the art. For example, the above described methods for introducing the anti-BACE molecules into a cell can also be used for introducing the molecules into a mammal.

The terms ‘pharmaceutical composition’ or ‘medicament’ or ‘use for the manufacture of a medicament to treat’ relate to a composition comprising an oligonucleotide comprising SEQ ID NO: 1 or 2 as described above and a pharmaceutically acceptable carrier or excipient (both terms can be used interchangeably) to treat Alzheimer's disease. Suitable carriers or excipients known to the skilled man are saline, Ringer's solution, dextrose solution, Hank's solution, fixed oils, ethyl oleate, 5% dextrose in saline, substances that enhance isotonicity and chemical stability, buffers and preservatives. Other suitable carriers include any carrier that does not itself induce the production of antibodies harmful to the individual receiving the composition such as proteins, polysaccharides, polylactic acids, polyglycolic acids, polymeric amino acids and amino acid copolymers.

The medicament can be administered to a mammal by any method known to those skilled in the art. Some examples of suitable modes of administration include oral and systemic administration. Systemic administration can be enteral or parenteral. Liquid or solid (e.g., tablets, gelatin capsules) formulations can be employed.

Parenteral administration of the medicament includes, for example intravenous, intramuscular, and subcutaneous injections. For instance, a molecule may be administered to a mammal by sustained release, as is known in the art. Sustained release administration is a method of drug delivery to achieve a certain level of the drug over a particular period of time. Other routes of administration include oral, topical, intrabronchial, or intranasal administration. For oral administration, liquid or solid formulations may be used. Some examples of formulations suitable for oral administration include tablets, gelatin capsules, pills, troches, elixirs, suspensions, syrups, and wafers. Intrabronchial administration can include an inhaler spray. For intranasal administration, administration of a molecule of the present invention can be accomplished by a nebulizer or liquid mist.

In a particular embodiment, the ‘medicament’ may be administered by a method close to the place of onset. In a particular embodiment, the medicament is delivered intrathecally. Intrathecal administration can for example be performed by means of surgically implanting a pump and running a catheter to the spine.

In another particular embodiment, the medicament is delivered intracerebroventricularly (ICV). ICV delivery may seem cumbersome at first sight, but is technically feasible and already operational for other chronic neurological indications. The discomfort of a single surgical intervention to position the pump may, in fact, greatly outweigh the benefits of this route of administration (lack of systemic side effects and immune response; controllable administration).

Generally, the medicament is administered so that the oligonucleotide comprising SEQ ID NO: 1 or 2 is given at a dose between 1 g/kg/day and 1000 μg/kg/day, more preferably between 10 μg/kg/day and 500 μg/kg/day, most preferably between 100 μg/kg/day and 500 μg/kg/day. Preferably a continuous infusion is used and includes the continuous subcutaneous delivery via an osmotic mini-pump.

In yet another embodiment, the invention provides the use of SEQ ID NO: 1 or 2 as a diagnostic marker for the presence of Alzheimer's disease in a subject. Thereto methods known in the art for the detection of SEQ ID NO: 1 or 2 are used. A common method is the use of quantitative real-time PCR (Q-PCR) which is herein further described in the materials and methods section. For diagnosis a body fluid of the mammal is taken. Suitable body fluids are blood or serum. A preferred body fluid is cerebrospinal fluid. It is envisaged that the absence or a reduced expression of SEQ ID NO: 1 or 2 is indicative for the presence or predisposition for Alzheimer's disease in a patient. In a preferred embodiment, SEQ ID NO: 1 is used for detecting the presence or predisposition of Alzheimer's disease.

### EXAMPLES

### 1) Increased Bace1 in Sporadic AD Brain

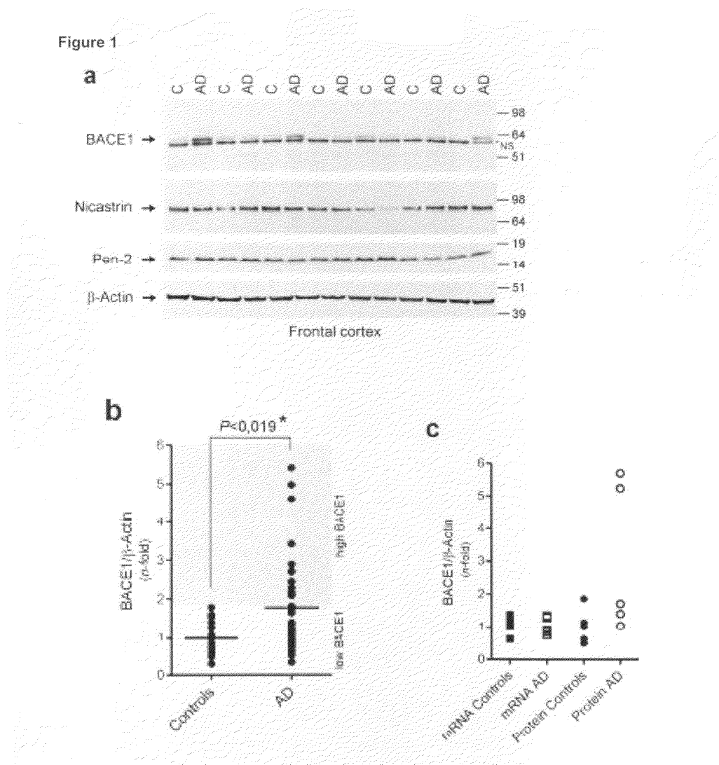

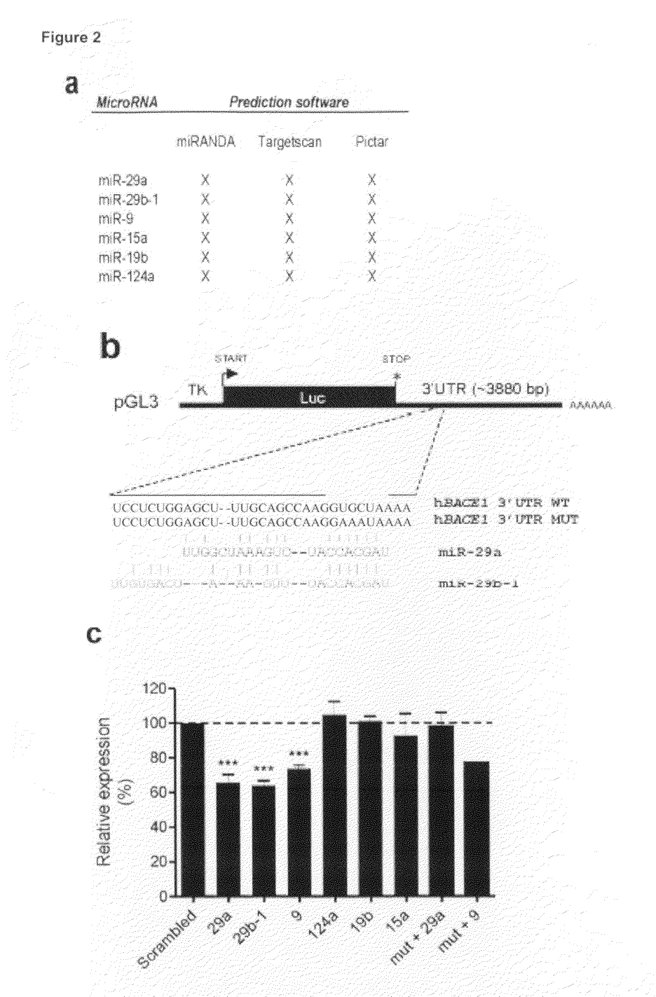

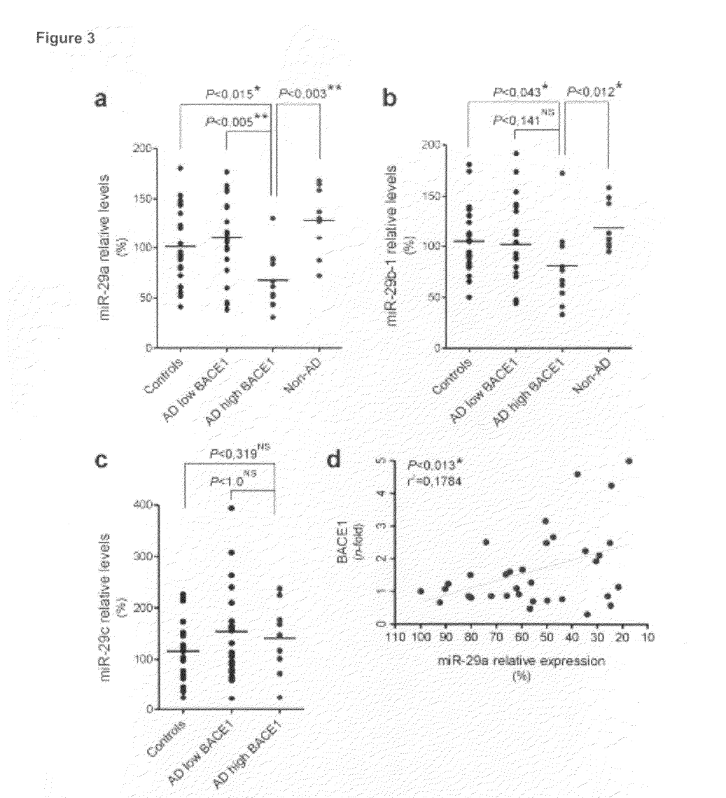

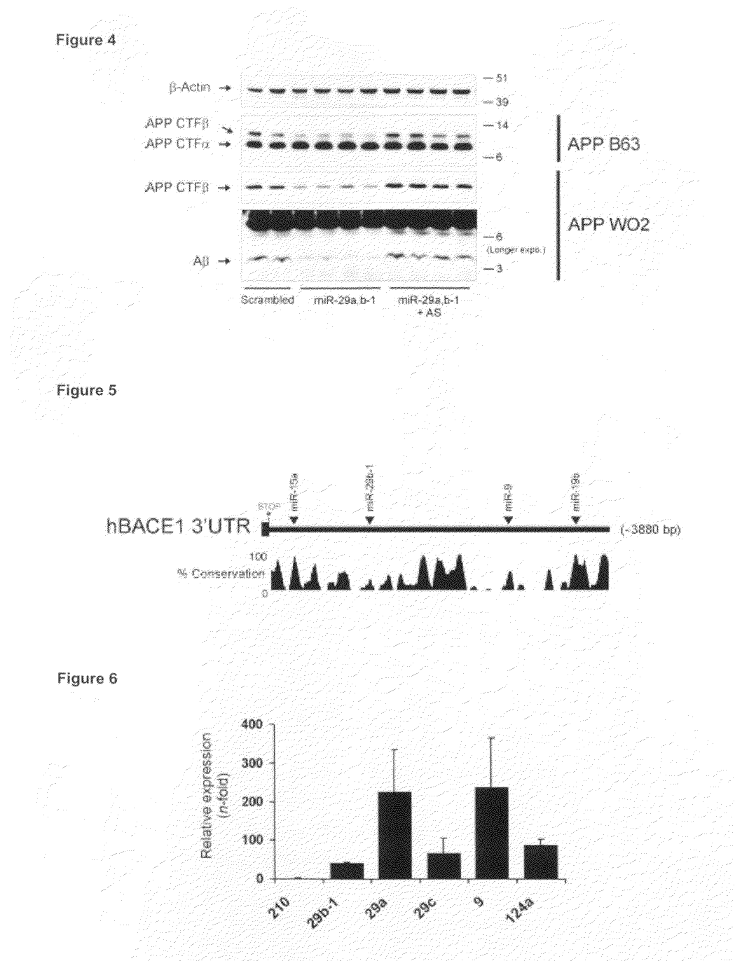

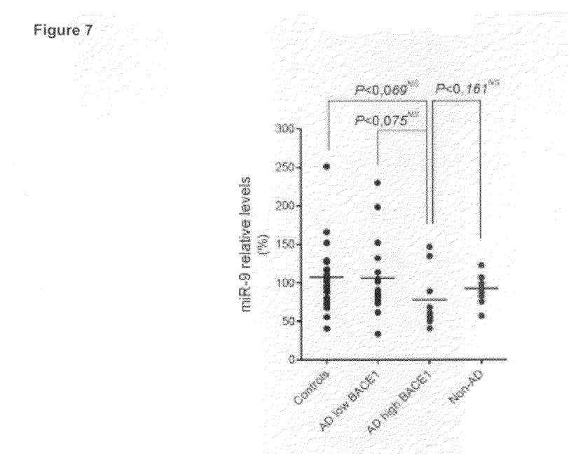

We analyzed a total of 55 frozen brain samples from 34 sporadic AD patients and 21 controls by Western blotting. Consistent with previous reports (Fukumoto et al., 2002; Holsinger et al., 2002; Sun et al., 2002; Yang et al., 2003), BACE1 protein was significantly increased in our sporadic AD patient samples (p=0.019, Mann Whitney test) (FIGS. 1a, b). This difference is contributed by a subgroup of approximately 30% of AD patients (11/34) that displays a BACE1 expression level which is 2 to 5 fold higher than in control samples (FIG. 1b). We used β-Actin as loading control and the expression of the γ-secretase components Nicastrin and Pen-2 as further internal controls, to show that these changes are specific for BACE1 (FIG. 1a). Consistent with previous data (Holsinger et al., 2002), mRNA levels for BACE1 remained essentially unchanged in a representative set of samples as shown by RT-PCR (compare mRNA controls to mRNA AD) (FIG. 1c), implying posttranscriptional regulation of BACE1 protein. Overall, these results suggest that BACE1 expression is affected at the posttranscriptional level in a subgroup of sporadic AD patients.

### 2) In Silico Analysis of the BACE1 3′UTR

To determine if miRNAs could target BACE1, we performed an in silico analysis using the Web-based prediction algorithms miRanda (John et al., 2004), Targetscan (Lewis et al., 2005; Lewis et al., 2003) and Pictar (Grun et al., 2005). We identified a surprisingly large number of potential miRNA binding sites within the 3′ UTR of BACE1 mRNA (FIG. 2a). These include target sites for miR-29a, miR-29b-1, miR-9, miR-15a, miR-19b and miR-124a. This 3′UTR is long (˜3880 bp) and appears evolutionary well conserved, in particular at the level of these candidate miRNA binding sites (FIG. 5). To validate these theoretical predictions experimentally, we generated a luciferase reporter construct containing the full predicted 3′UTR region of human BACE1 under the control of the human thymidine kinase promoter (FIG. 2b) and transiently co-transfected this construct with a series of synthetic miRNA precursors (pre-miRs) selected by our in silico analysis (FIG. 2a). Of all tested miRNAs, only miR-29a (p=0.008, Wilcoxon Signed-Rank test), miR-29b-1 (p=0.0313, Wilcoxon Signed-Rank test) and miR-9 (p=0.0313, Wilcoxon Signed-Rank test) affected significantly luciferase expression in Hela cells (FIG. 2c). Both miR-29a and miR-29b-1 target similar conserved binding sites within the BACE1 3′UTR (FIG. 2b). We mutated the conserved miR-29 “seed” sequence in the BACE1 3′UTR. This mutation abrogated BACE1 downregulation by miR-29a (FIG. 2c, bar indicated with mut+29a)

We confirmed by transient overexpression in human neuroblastoma SHSY-5Y cells the effects of miR-29a/b-1 on endogenous BACE1 expression providing further <<proof of concept>> (FIG. 2d). Western blot analysis of β-Actin as well as Nicastrin were used as normalization and internal controls respectively to show that these effects were specific for BACE1.

### 3) Changes in miR-29a/b-1 Correlate with BACE1 Expression in AD

In humans, miR-29a and miR-29b-1 are part of a larger gene family which includes miR-29c (http://microrna.sanger.ac.uk). MiR-29a and miR-29b-1 are however co-expressed as a cluster on chromosome 7 whereas miR-29c is located on chromosome 1. We could show that all three miR-29 family members, as well as miR-9, are highly expressed in human adult brain (FIG. 6), consistent with other reports (Barad et al., 2004; Hohjoh and Fukushima, 2007; Miska et al., 2004; Sempere et al., 2004). We next checked to what extent these 4 miRNAs were affected in the subgroup of sporadic AD patients with high BACE1 expression using quantitative RT-PCR. The ubiquitously expressed miR-16 was used as internal control to compare the different patient samples.

We found a significant reduction of miR-29a (p=0.015, Mann Whitney test) and miR-29b-1 (p=0.043, Mann Whitney test) in the subgroup of sporadic AD with high BACE1 levels when compared to controls or to patients with normal levels of BACE1 (indicated as “low” BACE1) (FIGS. 3a, b). A tendency to lower miR-9 levels was also observed in the high BACE1 patients, but this observation did not reach statistical significance (p=0.07, Mann Whitney test) (FIG. 7). Finally, miR-29c was not changed between controls and patient subgroups (p=0.319, Mann Whitney test) (FIG. 3c), indicating the specificity of our observations. We also measured the expression levels of non-relevant (i.e., not predicted to bind to the BACE1 3′UTR) miR-210 and miR-181c in 10 controls and 16 AD cases (with high and low BACE1) and observed no changes between these groups (data not shown). We finally verified miR-29a/b-1 levels in an additional group of 9 patients with non-AD dementia (Table 1), which displayed (FIGS. 3a and b) no significant changes compared to controls (p=0.094 for miR-29a and p=0.108 for miR-29b-1, Mann Whitney tests). Thus overall, we find that in the subgroup of sporadic AD patients with increased BACE1 expression, miR-29a and miR-29b-1 expression is significantly and specifically decreased.

A direct proof of the causal relationship between BACE1 expression and miR-29 cannot be provided in patients. However, we found a statistically significant (p=0.03, non-linear fit) correlation between BACE1 and miR-29a expression in the complete set of sporadic AD cases (34 patients) providing indirect support for this hypothesis (FIG. 3d). A relation between BACE1 and miR-29b-1 failed to reach statistical significance in this test (data not shown).

### 4) MiR-29a/b-1 Modulates BACE1 Activity and Aβ

We finally investigated (in a cell culture system) whether a causal relationship between miR-29a/b-1 expression and endogenous BACE1 activity exists. We used HEK293 cells stably expressing human APP Swedish form to target endogenous BACE activity. This type of cell line has been used frequently in the past to validate biochemical aspects of APP processing and Aβ metabolism. We show by Western blot analysis that levels of APP β-CTFs and Aβ peptide (the two major proteolytic fragments of BACE activity, FIG. 4c) are down-regulated in this cell culture system upon transient transfection with miR-29a/b-1. Importantly, these functional effects could be reversed with antisense anti-miR-29a/b-1 oligonucleotides, resulting again in a relative increase in Aβ generation. Thus loss of the suppressing activity of the miR-29a/b-1 cluster can lead to an increase in Aβ production in cell culture. In an independent set of experiments, we show by Western blot analysis that soluble “shed” APP-β as well as soluble Aβ is specifically reduced upon miR-29a and/or miR-29b-1 treatment. Here, soluble APP-α generated by α-secretase activity was used as internal control.

### 5) In Vivo Delivery of the microRNAs into the Brain of Mice

The aim of the experiment is to investigate and validate the role of the microRNA mmu-miR-29a (SEQ ID NO: 1) and microRNA mmu-miR-29b-1 (SEQ ID NO: 2) in the regulation of endogenous BACE1 expression in the mouse brain.

For this, chemically modified double-stranded RNA (ds-RNA, duplex form) oligonucleotides are custom synthesized as described (Krutzfeldt et al., 2005) Nature 438, 685) using the mature (underlined) miR-29a (SEQ ID NO: 1) and miR-29b-1 (SEQ ID NO: 2) sequences as templates:

mmu-miR-29a:
2-0 methyl- 5′ UAGCACCAUCUGAAAUCGGUU 3′

(SEQ ID NO: 14)

3′ dTuGUCGUGGUAGACUUUAGCC
mmu-miR-29b-1:
2-0 methyl- 5′ UAGCACCAUUUGAAAUCAGUGUU 3′

(SEQ ID NO: 15)

3′ dTuGUCGUGGUAAACUUUAGUCAC

Negative control (scrambled sequence):

(SEQ ID NO: 3)

| | |
|---|---|
| 2-0 methyl- | 5′ UUCACAAUGCGUUAUCGGAUGU 3′ |

(SEQ ID NO: 16)

| | |
|---|---|
| | 3′ dTuGAGUGUUACGCAAUAGCCU |
| SEQ ID NO: 1: | UAGCACCAUCUGAAAUCGGUU |
| SEQ ID NO: 2: | UAGCACCAUUUGAAAUCAGUGUU |

Technically, and consistent with previous methodologies (Wang et al., 2005) Neurosci Res, 53, 241), microRNA oligonucleotides (0.1-0.5 μg) are injected into new-born mouse (P2, where BACE1 is highly expressed) brain neurons using a Hamilton-type syringe that is designed for in vivo transfection.

Usually, three types of experimental controls are used: buffer-treated, control miRNA-treated (e.g., scrambled microRNA sequence, see above) and untreated.

For each newborn (P2) mouse, microRNA/transfection solution (e.g., Lipofectamine 2000 or ExGen 500) is directly injected into lateral ventricle to a depth of 2.0 mm approximately at the position of 1 mm to the right of and 1 mm posterior from Bregma. If necessary, Alcian-type blue dye is injected into control mice at P2 in order to define the site of injection of the microRNAs via histological examination of brain sections. Typically, analysis of brain extracts for BACE1 downregulation is performed 24-96 hours post-transfection.

In addition, injection of antisense probes (also known as “antagomiRs”) for microRNA miR-29a and miR-29b-1 are used to target and (functionally) knockdown specific microRNA species. AntagomiRs are single-stranded 31-33 nt 2-O-methyl (and cholesterol-modified) RNA oligonucleotides that can block the endogenous microRNA by forming a strong complex between the miRNA-RISC and the antagomiR, leading to loss of function of the microRNA in vivo.

For this, chemically modified single-stranded 31-33 nt RNA (ss-RNA) oligonucleotides are custom synthesized as described using as template the mouse miR-29a and miR-29b-1 mature sequences:

| | |
|---|---|
| mmu-miR-29a antagomiR: | |
| 5′ AACCGATTTCAGATGGTGCTA 3′ | (SEQ ID NO: 4) |
| mmu-miR-29b-1 antagomiR: | |
| 5′ AACACTGATTTCAAATGGTGCTA 3′ | (SEQ ID NO: 5) |

As above, these microRNA oligonucleotides are injected using a Hamilton-type syringe in the adult brain (into the ventricles) where endogenous miR-29a and miR-29b-1 are highly expressed. Typically, analysis of brain extracts for BACE1 upregulation is performed 24-96 hours post-transfection.

We investigate, in parallel, adapting an established protocol in which small RNA oligonucleotides can be specifically delivered in the brain and target complementary RNA strands. Recent data show that injecting small RNA oligos (similar in length to the mature microRNAs) in the brain can lead to efficient, widespread and specific mRNA targeting and gene knockdown (Smith et al., 2006). Technically, this is carried out with the same chemically modified microRNA oligonucleotides described which are continuously pumped into the right lateral ventricle of the adult mouse via a catheter surgically implanted through the skull and connected to an osmotic pump imbedded subcutaneously. By using this method, significant oligonucleotide concentrations can be achieved not only in the brain and brainstem but also in all levels of the spinal cord. As validation, the effects on endogenous target genes (like BACE1), Aβ and tau metabolism as well as neuronal survival is investigated using the abovementioned techniques. These techniques can easily be applied to normal as well as Alzheimer's disease mouse models known in the art.

The murine AD model used in the present invention over-expresses both mutated APP (APPSwe-KM670/671NL) and Psen1 (PS1-L166P) from a single genetic locus (“APP/PS1”) (Radde R et al (2006) EMBO Rep 7:940-946).

In another approach synthetic miR-29a and/or miR-29b-1 oligonucleotides are coupled to the RVG-9R peptide, as described in Kumar P. et al (2007) Nature 448: 39-43. RVG-9R is a chimeric peptide synthesized by adding nonamer arginine residues at the carboxy terminus of RVG (rabies virus glycoprotein). The sequence of the RVG-9R peptide is YTIWMPENPRPGTPCDIFTNSRGKRASNGGGGRRRRRRRRR (SEQ ID NO:6).

This 29-amino-acid peptide specifically binds to the acetylcholine receptor expressed by neuronal cells. This RVG-9R peptide is able to bind and transduce small RNA oligonucleotides to neuronal cells, resulting in efficient gene silencing. After intravenous injection into mice, RVG-9R delivers miR-29a and/or miR-29b-1 to the neuronal cells, resulting in specific gene silencing of BACE1 within the brain, but not in other organs. This leads to downregulation of BACE1, and per consequence decreased β-secretase activity and Aβ peptide levels. In yet another approach, the anti-BACE microRNA (miR-29a and/or miR-29b-1) is part of a hairpin precursor sequence. The anti-BACE microRNA or hairpin precursor is inserted into a vector, such as, for example, a recombinant vector. The vector may be, for example, a viral vector, such as an adenovirus vector or an adeno-associated virus (AAV) vector. Details regarding the generation, cloning and expression of small RNA oligonucleotides into AAV-based vectors are found in McBride J L et al (2008) Proc. Natl. Acad. Sciences 105(15):5868-73. As described in the latter reference, an AAV which encodes a hairpin for the anti-BACE1 microRNA oligonucleotide(s) is injected into the brain (ventricles or hippocampus). This is performed using the above-mentioned AD mouse model. The effects on BACE1 expression and AD pathology can be assessed up to four months after injection.

### Materials and Methods

### Patients and Cell Lines

The non-dementia (n=21), dementia (n=34) and non-AD dementia (n=9) patients were from the geriatric department of E. Roux Hospital at Limeil-Brevannes and the Lille CH&U Hospital, France (ADERMA network). They represent all patients who were hospitalized for various disorders and died at this hospital, excluding those whose family opposed autopsy, or for whom postmortem delay was more than 24 hours. Clinical data are detailed in Table 1. Cognitive status was evaluated using the Mini-Mental State Examination (“MMSE”) and the Clinical Dementing Rating (“CDR”) score. Clinical criteria for dementia were based on Diagnostic and Statistical Manual of Mental Disorders, 3rd ed, rev; for AD, National Institute of Neurological and Communicative Disorders and Stroke-Alzheimer's Disease and Related Disorders Association; for vascular dementia, National Institute of Neurological Disorders and Stroke Association Internationale pour la Recherche et l′Enseignement en Neurosciences; and for mixed dementia, Hachinski score. Clinical diagnosis was summarized as Alzheimer, AD (possible, probable), vascular dementia, mixed dementia (AD with a strong vascular involvement revealed by investigations), or dementia (for patients with an uncertain clinical diagnosis). Similarly, neuropathologic, (amyloid plaques, Braak stages of neurofibrillary degeneration, vascular pathology, other lesions), biochemical (tau, amyloid Abeta x-42 and x-40 and synucleinopathy) and genetic data (ApoE) for each patient are summarized in Table 1, as described in Delacourte et al, 1999; Delacourte et al, 2002; Deramecourt et al, 2006 (for references, see Table 1).

Blocks from the anterior temporal cortex were dissected from each case and snap frozen in liquid nitrogen.

Native SHSY-5Y cells and HEK293 stably expressing APP-Swedish form were cultured in DMEM/F12 medium as described (Hebert et al., 2006).

### Antibodies

Polyclonal APP B63.3(Hebert et al., 2006), monoclonal APP WO2 (epitope Aβ4-10, Heidelberg, Germany), polyclonal APP B54.4 (recognizes specifically the APP-β cleavage Swedish mutant epitope, a generous gift from B. Greenberg), monoclonal APP 6E10 (epitope Aβ1-16, Signet Laboratories), monoclonal β-Actin (Sigma-Aldrich), polyclonal BACE1 (cat#2253, Prosci, Inc.), monoclonal Nicastrin 9C3 (Esselens et al., 2004) and polyclonal Pen-2 B96(Hebert et al., 2006) were used.

### Protein Extraction and Western Blot Analysis

Cells were rinsed with cold PBS and lysed in buffer: 1% Triton X-100, 50 mM HEPES pH 7.6, 150 mM NaCl, 1 mM EDTA and complete protease inhibitors (Roche). DMEM/F12 medium was collected and spun at 1000 g for 5 min. Then, the supernatant was mixed with reducing loading buffer and heated at 70° C. for 10 min. Proteins from brain tissue were extracted using the mirVana PARIS kit (Ambion) following manufacturer's instructions. Immunoblot analysis was performed as described (Hebert et al., 2006).

Quantitative RT-PCR (mRNA)

Total RNA was extracted using the mirVana PARIS kit (Ambion) following manufacturer's instructions. RT-PCR as well as quantitative PCR was performed as described²⁶ using an ABI 7000 Sequence Detector. Human BACE1: 5′ CACCACCAACCTTCGTTTGC 3′ (SEQ ID NO:7) and 5′ AGTTCCCTGATGGTTTCTGGC 3′ (SEQ ID NO:8). Human β-Actin: 5′CACCCTGAAGTACCCCATGG 3′ (SEQ ID NO:9) and 5′ TGCCAGATTTTCTCCATGTCG 3′ (SEQ ID NO:10). Similar results were obtained using human GAPDH as internal control.

Quantitative RT-PCR (microRNA)

Each RT-PCR reaction was performed triplicates. The Taqman microRNA reverse transcription kit (Applied Biosystems) and the Taqman Universal PCR master mix (Applied Biosystems) were used. The quantitative PCR procedures were carried out following manufacturer's instructions provided with the Taqman microRNA assays (Applied Biosystems). Relative expression was calculated by using the comparative CT method. Out of three candidate reference genes (RNU19, RNU48 and hsa-miR-16), miR-16 was selected using the GeNorm software (worldwideweb://medgen.ugent.be/˜vdesomp/genorm).

### Probes for Quantitative RT-PCR (5′→3′)

Genes indicated with an “*” also recognize
mouse sequences.

| Gene | Probe sequence |
|---|---|
| hsa-miR-29a* | UAGCAGCAUCUGAAAUCGGUU |
| | (SEQ ID NO: 1) |
| hsa-miR-29b-1* | UAGCACCAUUUGAAAUCAGUGUU |
| | (SEQ ID NO: 2) |
| hsa-miR-16* | UAGCAGCACGUAAAUAUUGGCG |
| | (SEQ ID NO: 11) |

### Transfections, DNA Cloning and Luciferase Assays

200,000 Hela cells were plated in 6 well plates. The next day, cells were transfected with 25-75 nM of pre-miRs (Ambion) or a scrambled sequence (Ambion) using lipofectamine 2000 following manufacturer's instructions. 48 hours post-transfection, cells were processed for immunoblot analysis. The 3′UTR of human BACE and the TK promoter were amplified from human chromosomal DNA and cloned into the pGL3-luciferase basic vector (Promega). Inserts were confirmed by sequencing. The BACE1 luciferase mutant construct was generated using the QuickChange II K-XL Site-Directed Mutagenesis kit (Stratagene) according to manufacturer's instructions.

For the luciferase assays, 75 nM of pre-miRs (Ambion) were co-transfected with the sensor vector and the Renilla control vector (Promega). 26-28 hours post-transfection, the measurements were performed using the Dual luciferase reporter assay kit (Promega).

### Statistical Methodologies

BACE1 densitometric quantifications were performed using Image Quant software (Amersham Biosciences). Statistical significance was determined using two-tailed Mann-Whitney or Wilcoxon signed rank tests (as indicated in the text). The following regression model was used to calculate the correlation between BACE1 and miR-29a (which assumes a non-linear fit): BACE1=a+b1*X+b2*X̂2 where X=miR-29a and a=3.1017, b1=-0.03726 and b2=0.000157. Note that a non-parametric Pearson's test using the same values also gave significant correlation between BACE1 and miR-29a (p=0.013, two-tailed, R²=0.1784) (not shown).

### REFERENCES

Ambros, V. (2004) The functions of animal microRNAs. Nature, 431, 350-355.
Barad, O., Meiri, E., Avniel, A., Aharonov, R., Barzilai, A., Bentwich, I., Einav, U., Gilad, S., Hurban, P., Karov, Y., Lobenhofer, E. K., Sharon, E., Shiboleth, Y. M., Shtutman, M., Bentwich, Z. and Einat, P. (2004) MicroRNA expression detected by oligonucleotide microarrays: system establishment and expression profiling in human tissues. Genome Res, 14, 2486-2494.
Bartel, D. P. (2004) MicroRNAs: genomics, biogenesis, mechanism, and function. Cell, 116, 281-297.
Delacourte A., David J. P., Sergeant N., Buee L., Wattez A., Vermersch P., Ghozali F., Fallet-Bianco C., Pasquier F., Lebert F., Petit H. and Di Menza C. (1999) The biochemical pathway of neurofibrillary degeneration in aging and Alzheimer's disease. Neurology 52, 1158-1165.
Delacourte A., Sergeant N., Champain D., Wattez A., Maurage C. A., Lebert F., Pasquier F. and David J. P. (2002) Nonoverlapping but synergetic tau and APP pathologies in sporadic Alzheimer's disease. Neurology 59, 398-407.
Deramecourt V., Bombois S., Maurage C. A., Ghestem A., Drobecq H., Vanmechelen E., Lebert F., Pasquier F. and Delacourte A. (2006) Biochemical staging of synucleinopathy and amyloid deposition in dementia with Lewy bodies. J Neuropathol Exp Neurol 65, 278-288.
Esselens, C., Oorschot, V., Baert, V., Raemaekers, T., Spittaels, K., Serneels, L., Zheng, H., Saftig, P., De Strooper, B., Klumperman, J. and Annaert, W. (2004) Presenilin 1 mediates the turnover of telencephalin in hippocampal neurons via an autophagic degradative pathway. J Cell Biol, 166, 1041-1054.
Fukumoto, H., Cheung, B. S., Hyman, B. T. and Irizarry, M. C. (2002) Beta-secretase protein and activity are increased in the neocortex in Alzheimer disease. Arch Neurol, 59, 1381-1389.
Grun, D., Wang, Y. L., Langenberger, D., Gunsalus, K. C. and Rajewsky, N. (2005) microRNA target predictions across seven Drosophila species and comparison to mammalian targets. PLoS Comput Biol, 1, e13.
Hebert, S. S., Serneels, L., Tolia, A., Craessaerts, K., Derks, C., Filippov, M. A., Muller, U. and De Strooper, B. (2006) Regulated intramembrane proteolysis of amyloid precursor protein and regulation of expression of putative target genes. EMBO Rep, 7, 739-745.
Hohjoh, H. and Fukushima, T. (2007) Expression profile analysis of microRNA (miRNA) in mouse central nervous system using a new miRNA detection system that examines hybridization signals at every step of washing. Gene, 391, 39-44.
Holsinger, R. M., McLean, C. A., Beyreuther, K., Masters, C. L. and Evin, G. (2002) Increased expression of the amyloid precursor beta-secretase in Alzheimer's disease. Ann Neurol, 51, 783-786.
John, B., Enright, A. J., Aravin, A., Tuschl, T., Sander, C. and Marks, D. S. (2004) Human MicroRNA targets. PLoS Biol, 2, e363.
Krutzfeldt, J., Rajewsky, N., Braich, R., Rajeev, K. G., Tuschl, T., Manoharan, M. and Stoffel, M. (2005) Silencing of microRNAs in vivo with ‘antagomirs’. Nature, 438, 685-689.
Lewis, B. P., Burge, C. B. and Bartel, D. P. (2005) Conserved seed pairing, often flanked by adenosines, indicates that thousands of human genes are microRNA targets. Cell, 120, 15-20.
Lewis, B. P., Shih, I. H., Jones-Rhoades, M. W., Bartel, D. P. and Burge, C. B. (2003) Prediction of mammalian microRNA targets. Cell, 115, 787-798.
Mehler, M. F. and Mattick, J. S. (2006) Non-coding RNAs in the nervous system. J Physiol, 575, 333-341.
Miska, E. A., Alvarez-Saavedra, E., Townsend, M., Yoshii, A., Sestan, N., Rakic, P., Constantine-Paton, M. and Horvitz, H. R. (2004) Microarray analysis of microRNA expression in the developing mammalian brain. Genome Biol, 5, R68.
Nutt, J. G., Burchiel, K. J., Comella, C. L., Jankovic, J., Lang, A. E., Laws, E. R., Jr., Lozano, A. M., Penn, R. D., Simpson, R. K., Jr., Stacy, M. and Wooten, G. F. (2003) Randomized, double-blind trial of glial cell line-derived neurotrophic factor (GDNF) in PD. Neurology, 60, 69-73.
Sempere, L. F., Freemantle, S., Pitha-Rowe, I., Moss, E., Dmitrovsky, E. and Ambros, V. (2004) Expression profiling of mammalian microRNAs uncovers a subset of brain-expressed microRNAs with possible roles in murine and human neuronal differentiation. Genome Biol, 5, R13.
Smith, R. A., Miller, T. M., Yamanaka, K., Monia, B. P., Condon, T. P., Hung, G., Lobsiger, C. S., Ward, C. M., McAlonis-Downes, M., Wei, H., Wancewicz, E. V., Bennett, C. F. and Cleveland, D. W. (2006) Antisense oligonucleotide therapy for neurodegenerative disease. J Clin Invest, 116, 2290-2296.
Sun, A., Koelsch, G., Tang, J. and Bing, G. (2002) Localization of beta-secretase memapsin 2 in the brain of Alzheimer's patients and normal aged controls. Exp Neurol, 175, 10-22.
Wang, Y. L., Liu, W., Wada, E., Murata, M., Wada, K. and Kanazawa, I. (2005) Clinico-pathological rescue of a model mouse of Huntington's disease by siRNA. Neurosci Res, 53, 241-249.
Yang, L. B., Lindholm, K., Yan, R., Citron, M., Xia, W., Yang, X. L., Beach, T., Sue, L., Wong, P., Price, D., Li, R. and Shen, Y. (2003) Elevated beta-secretase expression and enzymatic activity detected in sporadic Alzheimer disease. Nat Med, 9, 3-4.

1. A medicament comprising:
an oligonucleotide of less than 100 nucleotides comprising SEQ ID NO: 1 and/or SEQ ID NO: 2 or a modified oligonucleotide of less than 100 nucleotides comprising a modified SEQ ID NO: 1 and/or SEQ ID NO: 2 in an amount useful to treat Alzheimer's disease.

2. A method of treating a subject suffering from or at risk of suffering from Alzheimer's disease, the method comprising:
administering to the subject an oligonucleotide of less than 100 nucleotides comprising SEQ ID NO: 1 and/or SEQ ID NO: 2 or a modified oligonucleotide of less than 100 nucleotides comprising a modified SEQ ID NO: 1 and/or SEQ ID NO: 2 in an amount useful to treat Alzheimer's disease.

3. The method according to claim 2, wherein said oligonucleotide is delivered to the subject intrathecally.

4. The method according to claim 2, wherein said oligonucleotide is delivered to the subject intravenously.

5. The method according to claim 2, wherein said oligonucleotide is delivered to the subject intra-cerebroventricularly.

6. A method of diagnosing Alzheimer's disease in a subject, the method comprising:
taking a sample selected from the group consisting of cerebrospinal fluid, blood, serum, and any combination thereof from the subject,
analyzing said sample for the presence of SEQ ID NO: 1 and/or SEQ ID NO: 2 wherein the absence or reduced expression of SEQ ID NO:1 or SEQ ID NO:2 in comparison to a standard is indicative of Alzheimer's disease in the subject.

7. The method according to claim 6, wherein the analysis is by quantitative real time PCR.

8. The method according to claim 6, wherein the analysis is for SEQ ID NO:1.

9. The method according to claim 6, wherein the analysis is for SEQ ID NO:2.

10. The method according to claim 6, wherein the sample is cerebrospinal fluid.

11. The method according to claim 10, wherein the analysis is by quantitative real time PCR.

12. The method according to claim 11, wherein the analysis is for SEQ ID NO:1.

13. The method according to claim 11, wherein the analysis is for SEQ ID NO:2.


SEQUENCE LISTING (16 sequences)

SEQ ID NO: 1 - RNA, 21 nt, Homo sapiens
uagcaccaucugaaaucgguu

SEQ ID NO: 2 - RNA, 23 nt, Homo sapiens
uagcaccauuugaaaucaguguu

SEQ ID NO: 3 - RNA, 22 nt, Homo sapiens
uucacaaugcguuaucggaugu

SEQ ID NO: 4 - DNA, 21 bp, Homo sapiens
aaccgatttcagatggtgcta

SEQ ID NO: 5 - DNA, 23 bp, Homo sapiens
aacactgatttcaaatggtgcta

SEQ ID NO: 6 - protein, 41 aa, Rabies virus
YTIWMPENPRPGTPCDIFTNSRGKRASNGGGGRRRRRRRRR

SEQ ID NO: 7 - DNA, 20 bp, Homo sapiens
caccaccaaccttcgtttgc

SEQ ID NO: 8 - DNA, 21 bp, Homo sapiens
agttccctgatggtttctggc

SEQ ID NO: 9 - DNA, 20 bp, Homo sapiens
caccctgaagtaccccatgg

SEQ ID NO: 10 - DNA, 21 bp, Homo sapiens
tgccagattttctccatgtcg

SEQ ID NO: 11 - RNA, 22 nt, Homo sapiens
uagcagcacguaaauauuggcg

SEQ ID NO: 12 - RNA, 32 nt, Artificial Sequence
  hBACE1 3′UTR Wild-type
uccucuggagcuuugcagccaaggugcuaaaa

SEQ ID NO: 13 - RNA, 32 nt, Artificial Sequence
  hBACE1 3′UTR Mutant
uccucuggagcuuugcagccaaggaaauaaaa

SEQ ID NO: 14 - RNA, 19 nt, Artificial Sequence
  mRNA with a 3′dTu
ccgauuucagauggugcug

SEQ ID NO: 15 - RNA, 21 nt, Artificial Sequence
  mRNA with a 3′dTu
cacugauuucaaauggugcug

SEQ ID NO: 16 - RNA, 19 nt, Artificial Sequence
  mRNA with a 3′dTu
uccgauaacgcauugugag